(19) United States
(12) Patent Application Publication
(10) Pub. No.: US 2025/0099712 A1
(43) Pub. Date: Mar. 27, 2025

(54) SLEEP MANAGEMENT SYSTEM AND SLEEP MANAGEMENT METHOD

(71) Applicant: SAMSUNG ELECTRONICS CO., LTD., Suwon-si (KR)

(72) Inventors: Sunok KIM, Suwon-si (KR); Yuhyun AN, Suwon-si (KR); Junho LEE, Suwon-si (KR); Bosung JUNG, Suwon-si (KR)

(73) Assignee: SAMSUNG ELECTRONICS CO., LTD., Suwon-si (KR)

(21) Appl. No.: 18/920,303

(22) Filed: Oct. 18, 2024

Related U.S. Application Data
(63) Continuation of application No. PCT/KR2024/012063, filed on Aug. 13, 2024.

(30) Foreign Application Priority Data
Sep. 27, 2023 (KR) 10-2023-0131110

(51) Int. Cl. A61M 21/02; A61M 21/00

(52) U.S. Cl. CPC A61M 21/02; A61M 2021/0027; A61M 2021/0044; A61M 2205/3303; A61M 2205/50

(57) ABSTRACT

A sleep management system includes a plurality of sensors obtaining user data; a hub device configured to preprocess the user data obtained by the plurality of sensors; and a user device configured to obtain the preprocessed user data from the hub device. The user device determines a stability level of the user based on the preprocessed user data, provides a sleep induction program corresponding to the stability level of the user through a user interface, monitors a change in the stability level of the user according to the providing of the sleep induction program, and provides feedback information corresponding to the change in the stability level of the user through the user interface.

### CROSS-REFERENCE TO RELATED APPLICATIONS

This application is a by-pass continuation of International Application No. PCT/KR2024/012063, filed on Aug. 13, 2024, which is based on and claims priority to Korean Patent Application No. 10-2023-0131110, filed in the Korean Intellectual Property Office on Sep. 27, 2023, the disclosures of which are incorporated by reference herein in their entireties.

### BACKGROUND

### 1. Field

The disclosure relates to a sleep management system and sleep management method.

### 2. Description of Related Art

Sleep is an essential factor for a person's health and well-being as it plays a variety of roles, including repairing the body, forming new memories, staying focused, and removing waste products accumulated in the brain. Maintaining quality sleep is very important for humans to live healthy lives.

These days, a lot of people suffer from insomnia. There are various types of insomnia. For example, insomnia includes a sleep initiation disorder wherein a person has difficulty falling asleep or takes a long time to fall asleep. When sleep initiation disorder occurs, a person may feel quite stressed, which may lead to various pathological symptoms.

There are many different ways to help a user initiate sleeping, but a technology to automatically provide a suitable solution for the user does not exist.

### SUMMARY

Provided is a sleep management system and sleep management method for automatically providing a suitable sleep induction program for the user.

Further provided is a sleep management system and sleep management method which may provide proper feedback by monitoring a stability level of the user and automatically provide and terminate a sleep induction program.

According to an aspect of the disclosure, a sleep management system includes: a sensor configured to collect data regarding a user; a hub device configured to preprocess the data obtained by the sensor; and a user device comprising: at least one memory storing one or more instructions; and at least one processor configured to execute the one or more instructions, wherein the one or more instructions, when executed by the at least one processor of the user device, are configured to cause the user device to: obtain the preprocessed data from the hub device, identify a stability level of the user based on the preprocessed data, provide, through a user interface, a sleep induction program corresponding to the stability level of the user, monitor a change in the stability level of the user contemporaneously with the provision of the sleep induction program, and provide, through the user interface, feedback information corresponding to the change in the stability level of the user.

The one or more instructions, when executed by the at least one processor of the user device, may be further configured to cause the user device to: provide the sleep induction program by providing, based on the stability level of the user, a first sleep induction program configured to guide the user to physically relax or a second sleep induction program configured to guide the user to mentally relax.

The one or more instructions, when executed by the at least one processor of the user device, may be further configured to cause the user device to: based on the stability level of the user being lower than a reference level, provide the first sleep induction program, and based on the stability level of the user being equal to or higher than the reference level, provide the second sleep induction program.

The one or more instructions, when executed by the at least one processor of the user device, may be further configured to cause the user device to: stop providing the second sleep induction program based on the stability level of the user being equal to or higher than the reference level for a preset period of time.

The preprocessed data may include at least one of a movement intensity of the user, a pressure level of a body portion of the user, a respiration rate of the user, and a heart rate of the user.

The one or more instructions, when executed by the at least one processor of the user device, may be further configured to cause the user device to: monitor the change in the stability level of the user based on the first sleep induction program or the second sleep induction program being provided by monitoring a change in at least one of the movement intensity, the pressure level, the respiration rate and the heart rate.

The one or more instructions, when executed by the at least one processor of the user device, may be further configured to cause the user device to: based on the first sleep induction program being provided, provide first feedback information configured to increase the change in the stability level of the user, and based on the second sleep induction program being provided, provide second feedback information configured to reduce the change in the stability level of the user.

The one or more instructions, when executed by the at least one processor of the user device, may be further configured to cause the user device to: obtain at least one reference range regarding at least one of the movement intensity of the user, the pressure level for the body portion of the user, the respiration rate of the user and the heart rate of the user from at least one memory, and identify the stability level of the user by comparing at least one of the movement intensity, the pressure level, the respiration rate and the heart rate with the at least one reference range.

The one or more instructions, when executed by the at least one processor of the user device, may be further configured to cause the user device to: control the user interface to provide the sleep induction program and the feedback information as at least one of visual information and audio information.

The one or more instructions, when executed by the at least one processor of the user device, may be further configured to cause the user device to: generate control information for providing the sleep induction program and the feedback information through at least one of a display device and an audio output device, and transmit the control information to a server.

According to an aspect of the disclosure, a sleep management method includes: obtaining data regarding a user through a sensor; preprocessing the data by a hub device; identifying, by a user device, a stability level of the user based on the preprocessed data; providing, by the user device, a sleep induction program corresponding to the stability level of the user; monitoring, by the user device, a change in the stability level of the user contemporaneously with the providing the sleep induction program; and providing, by the user device, feedback information corresponding to the change in the stability level of the user.

The providing the sleep induction program may include providing, based on the stability level of the user, a first sleep induction program configured to guide the user to physically relax or a second sleep induction program configured to guide the user to mentally relax.

The providing the sleep induction program may further include: based on the stability level of the user being lower than a reference level, providing the first sleep induction program; and based on the stability level of the user being equal to or higher than the reference level, providing the second sleep induction program.

The sleep management method may further include: based on the stability level of the user maintained being equal to or higher than the reference level for a preset period of time, terminating the providing of the second sleep induction program.

The preprocessed data may include at least one of a movement intensity of the user, a pressure level of a body portion of the user, a respiration rate of the user, and a heart rate of the user.

According to an aspect of the disclosure, a non-transitory computer readable medium having instructions stored therein, which when executed by at least one processor cause the at least one processor to execute a sleep management method including: obtaining data regarding a user through a sensor; preprocessing the data by a hub device; identifying, by a user device, a stability level of the user based on the preprocessed data; providing, by the user device, a sleep induction program corresponding to the stability level of the user; monitoring, by the user device, a change in the stability level of the user contemporaneously with the providing the sleep induction program; and providing, by the user device, feedback information corresponding to the change in the stability level of the user.

With regard to the sleep management method executed based on the instructions stored in the non-transitory computer readable medium, the providing the sleep induction program comprises providing, based on the stability level of the user, a first sleep induction program configured to guide the user to physically relax or a second sleep induction program configured to guide the user to mentally relax.

With regard to the sleep management method executed based on the instructions stored in the non-transitory computer readable medium, the providing the sleep induction program may further include: based on the stability level of the user being lower than a reference level, providing the first sleep induction program; and based on the stability level of the user being equal to or higher than the reference level, providing the second sleep induction program.

With regard to the sleep management method executed based on the instructions stored in the non-transitory computer readable medium, the sleep management method may further include: based on the stability level of the user maintained being equal to or higher than the reference level for a preset period of time, terminating the providing of the second sleep induction program.

With regard to the sleep management method executed based on the instructions stored in the non-transitory computer readable medium, the preprocessed data may include at least one of a movement intensity of the user, a pressure level of a body portion of the user, a respiration rate of the user, and a heart rate of the user.

The sleep management system and sleep management method in the disclosure may automatically provide a sleep induction program suitable for the user.

The sleep management system and sleep management method in the disclosure may provide proper feedback by monitoring the stability level of the user and automatically provide and terminate the sleep induction program. Accordingly, they may provide effective help for initiating sleep of the user.

### BRIEF DESCRIPTION OF THE DRAWINGS

The above and other aspects and features of certain embodiments of the present disclosure will be more apparent from the following description taken in conjunction with the accompanying drawings, in which:

FIG. 1 illustrates a network of a sleep management system, according to an embodiment;

FIG. 2 schematically illustrates a structure of a sleep management system, according to an embodiment;

FIG. 3 illustrates a control block diagram of a sleep management system, according to an embodiment;

FIGS. 4 and 5 illustrate various examples of different layouts of sensors included in a sleep management system;

FIG. 6 illustrates a method of processing data obtained from a plurality of sensors;

FIG. 7 is a flowchart for schematically describing an example of a sleep management method performed by a user device;

FIG. 8 is a flowchart describing the sleep management method as described in FIG. 7 in more detail;

FIGS. 9 and 10 illustrate a sleep management method performed by a sleep management system, according to an embodiment;

FIGS. 11 and 12 illustrate a sleep management method performed by a sleep management system, according to another embodiment;

FIG. 13 illustrates an example of a screen for user state information provided through at least one of a user device and a home appliance;

FIG. 14 illustrates an example of feedback information provided based on a stability level of a user when a sleep induction program is provided;

FIG. 15 illustrates another example of feedback information provided based on a stability level of a user when a sleep induction program is provided; and

FIGS. 16, 17 and 18 illustrate various examples of controlling home appliances to provide a sleep induction program and feedback information.

### DETAILED DESCRIPTION

It is understood that various embodiments of the disclosure and associated terms are not intended to limit technical features herein to particular embodiments, but encompass various changes, equivalents, or substitutions.

Like reference numerals may be used for like or related elements throughout the drawings.

The singular form of a noun corresponding to an item may include one or more items unless the context states otherwise.

Throughout the specification, “A or B”, “at least one of A and B”, “at least one of A or B”, “A, B or C”, “at least one of A, B and C”, and “at least one of A, B, or C” may each include any one or all the possible combinations of A, B and C.

The expression “and/or” is interpreted to include a combination or any of the associated elements. For example, the expression “A, B and/or C” may include one of A, B, and C or any combination thereof.

Terms like “first”, “second”, etc., may be simply used to distinguish an element from another, without limiting the elements in a certain sense (e.g., in terms of importance or order).

When an element is mentioned as being “coupled” or “connected” to another element with or without an adverb “functionally” or “operatively”, it means that the element may be connected to the other element directly (e.g., wiredly), wirelessly, or through a third element.

It will be further understood that the terms “comprise” and/or “comprising,” when used in this specification, specify the presence of stated features, integers, steps, operations, elements, parts or combinations thereof, but do not preclude the possible presence or addition of one or more other features, integers, steps, operations, elements, components, and/or groups thereof.

When an element is mentioned as being “connected to”, “coupled to”, “supported on” or “contacting” another element, it includes not only a case that the elements are directly connected to, coupled to, supported on or contact each other but also a case that the elements are connected to, coupled to, supported on or contact each other through a third element.

Throughout the specification, when an element is mentioned as being located “on” another element, it implies not only that the element is abut on the other element but also that a third element exists between the two elements.

A sleep management system according to various embodiments will now be described in detail in connection with the accompanying drawings.

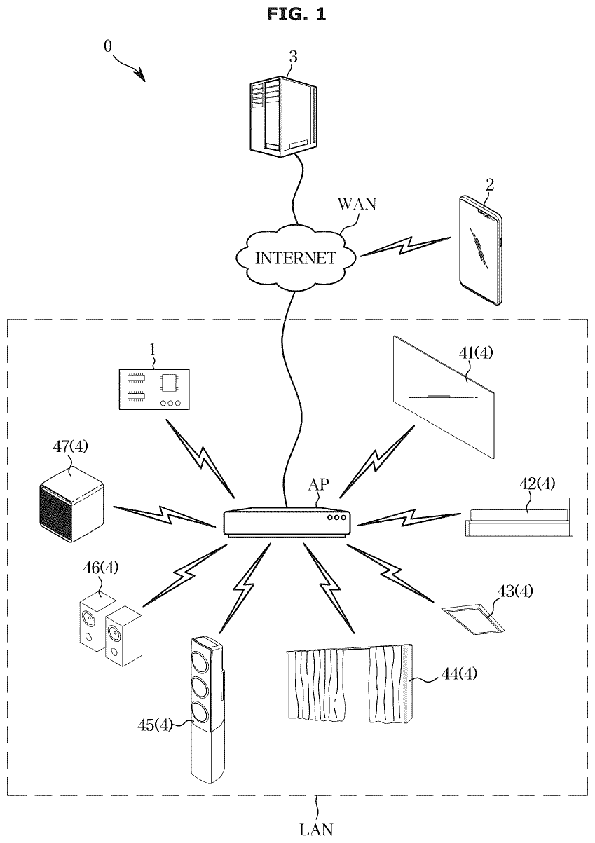

FIG. 1 illustrates a network of a sleep management system 0, according to an embodiment.

Referring to FIG. 1, the sleep management system 0 in the embodiment may include a hub device 1, a user device 2, a server 3 and/or home appliances 4.

The hub device 1 may include a communication module capable of communicating with the user device 2, the server 3 and/or the home appliances 4, at least one processor for processing data, and at least one memory that stores a program for controlling operation of the hub device 1.

The hub device 1 may process data obtained from one or more sensors, and obtain the processed data. In an embodiment, the hub device 1 may use a machine learning model to process data collected from the plurality of sensors.

The hub device 1 may transmit the processed data to the user device 2. For example, the hub device 1 may transmit the processed data to the user device 2, not through the server 3, but by direct communication.

The home appliances 4 may include various types of electronic products. For example, the home appliances 4 may include at least one of a display device 41, a furniture control device 42, a lighting device 43, an automatic curtain open/close device 44, an air conditioner 45, a speaker 46 and an air purifier 47. The aforementioned home appliances are merely examples, and other various types of electronic products such as a clothes care apparatus in addition to the aforementioned home appliance products may also be included in the home appliances 4.

The home appliance 4 may be controlled remotely by the user device 2 and the server 3.

The furniture control device 42 may include an actuator that may change a posture of the user by changing the structure of the furniture and/or a vibration element that may transmit vibration to the user who lies or sits on the furniture. For example, the furniture control device 42 may include an actuator that is able to control a reclination angle of a recliner bed, a recliner chair and/or a recliner sofa.

The lighting device 43 may include a light source with a controllable intensity and/or color of light.

The automatic curtain open/close device 44 may include an actuator for automatically opening or closing a curtain.

The server 3 may include a communication module for communicating with the hub device 1, the user device 2 and/or the home appliance 4.

The server 3 may include at least one processor that may process data received from the hub device 1, the user device 2 and/or the home appliances 4, and at least one memory that may store a program for processing data or processed data.

The server 3 may be implemented with various computing devices such as a workstation, a cloud, a data drive, a data station, etc. The server 3 may be implemented with one or more servers physically or logically classified based on function, sub-configuration of the function or data, and may transmit or receive data through inter-server communication and process the data.

The server 3 may perform functions of storing and/or managing a user account, registering the hub device 1, the user device 2 and/or the home appliance 4 by associating them with the user account, and managing or controlling the registered hub device 1 and the home appliance 4. For example, the user may access the server 3 through the user device 2 to create a user account. The user account may be identified by an identity (ID) and a password created by the user. The user may access the server 3 through the user device 2 to manage the user account.

The server 3 may register the hub device 1, the user device 2 and/or the home appliance 4 with the user account, according to a preset procedure. For example, the server 3 may connect identification information (e.g., a serial number, a media access control (MAC) address, etc.) of the hub device 1 to the user account to register, manage and control the hub device 1. Likewise, the server 3 may register the user device 2 and the home appliance 4 with the user account and control them. The server 3 may receive various information from the hub device 1, the user device 2 and/or the home appliance 4 registered with the user account.

The server 3 may include a plurality of servers for performing the same and/or different operations. For example, the server 3 may include a first server and a second server. The first server may create and/or manage user account information, and register and/or manage information about the hub device 1, the user device 2 and/or the home appliance 4 with the user account. The second server may receive registration information of the user device 2 and the home appliance 4 from the first server to control the user device 2 and/or the home appliance 4. The second server may perform a function of managing the hub device 1 and the home appliance 4 registered in the first server on behalf of the first server. The number of the servers 3 is not limited to the above example.

The user device 2 may include a communication module for communicating with the hub device 1, the server 3 and/or the home appliance 4. The user device 2 may include a user interface for receiving user inputs or outputting information for the user. The user device 2 may include at least one processor for controlling operation of the user device 2 and at least one memory for storing a program for controlling the operation of the user device 2.

The user device 2 may be carried by the user or placed at the user's home or office. The user device 2 may include a personal computer, a terminal, a mobile phone, a smart phone, a handheld device, a wearable device and/or a display device. The user device 2 is not limited to the above example.

In the memory of the user device 2, a program, software and/or an application for processing data received from the hub device 1 may be stored. The program, the software and/or the application may be sold in a state of being installed in the user device 2, or may be downloaded from the server 3 and stored in the user device 2.

When the program, the software and/or the application installed in the user device 2 is executed by the user, the user device 2 may access the server 3 and create a user account, and register the hub device 1 and/or the home appliance 4 by communicating with the server 3 based on the login user account.

For example, when the home appliance 4 is operated to access the server 3 according to a procedure guided in the application installed in the user device 2, the server 3 may register the home appliance 4 with the user account by registering the identification information (e.g., a serial number or a MAC address) of the home appliance 4 with the user account. The home appliance 4 may also be registered with the user account in the similar manner. Other information than the serial number or MAC address of the device to identify the device may be used for the information required to register the device such as the home appliance 4 with the user account.

The user device 2 may receive various information from the hub device 1 and the home appliance 4 registered with the user account directly or through the server 3.

A network may include both a wired network and a wireless network. The wired network may include a cable network or a telephone network, and the wireless network may include any network that transmits or receives signals in radio waves. The wired network and the wireless network may be connected to each other.

The network may include a wide area network (WAN) such as the Internet, a local area network (LAN) formed around an access point (AP), and a short-range wireless network without an AP. The short-range wireless network may include bluetooth (IEEE 802.15.1), Zigbee (IEEE 802.15.4), wireless fidelity (Wi-Fi) direct, near field communication (NFC), Z-wave, etc., without being limited thereto.

The AP may connect the hub device 1, the user device 2 and/or the home appliance 4 to the WAN connected to the server 3. The hub device 1, the user device 2 and/or the home appliance 4 may be connected to the server 3 through the WAN.

The AP may use wireless communication such as Wi-Fi (IEEE 802.11), Bluetooth (IEEE 802.15.1), Zigbee (IEEE 802.15.4), etc., to communicate with the hub device 1, the user device 2 and/or the home appliance 4, and use wired communication to access the WAN, but the wireless communication scheme of the AP is not limited thereto.

In an embodiment, the hub device 1 may communicate with the user device 2 over a short-range wireless network without going through the AP.

For example, the hub device 1 may be connected to the user device 2 over a short-range wireless network (e.g., Wi-Fi direct, bluetooth or NFC). In another example, the hub device 1 may use the long-range wireless network (e.g., a cellular communication module) to be connected to the user device 2 through the WAN.

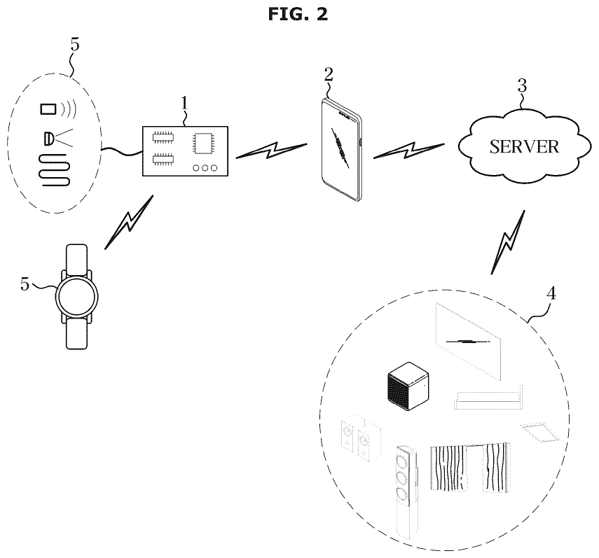

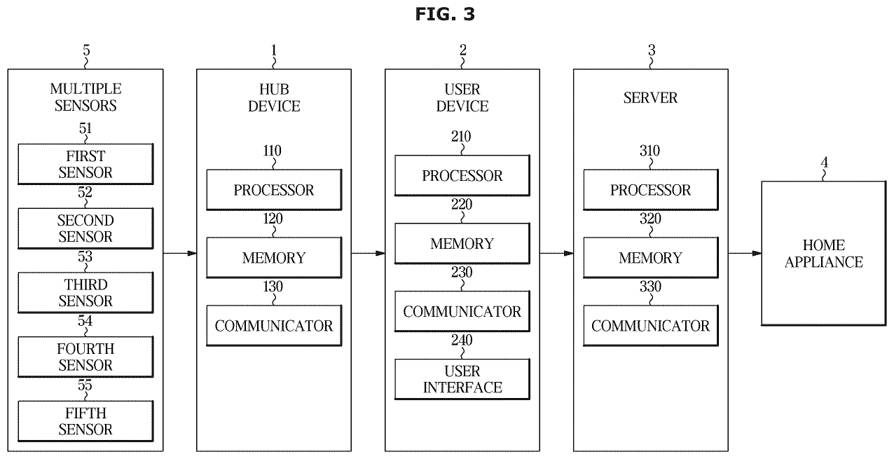

FIG. 2 schematically illustrates a structure of a sleep management system, according to an embodiment. FIG. 3 illustrates a control block diagram of a sleep management system, according to an embodiment.

Referring to FIGS. 2 and 3, the hub device 1 may obtain user data from a plurality of sensors 5.

The plurality of sensors 5 may include a first sensor 51, a second sensor 52, a third sensor 53, a fourth sensor 54 and a fifth sensor 55. The plurality of sensors 5 may each obtain user data. The plurality of sensors 5 may each transmit the obtained user data to the hub device 1. The user data may include various types of data related to the user's state.

Each of the first sensor 51, the second sensor 52, the third sensor 53, the fourth sensor 54 and the fifth sensor 55 may be one of a pressure sensor, an ultra-wideband (UWB) sensor, a radar sensor, an oxygen saturation sensor, an electrocardiogram (ECG) sensor and an acceleration sensor. For example, the first sensor 51 may be the pressure sensor, the second sensor 52 the UWB sensor, the third sensor 53 the radar sensor, the fourth sensor 54 the oxygen saturation sensor or the ECG sensor, and the fifth sensor 55 the acceleration sensor.

The plurality of sensors 5 may include at least two of a pressure sensor for obtaining pressure data corresponding to a pressure level for each body portion of the user, a UWB sensor for obtaining displacement data corresponding to displacement of the body that changes according to the user's breathing, an oxygen saturation sensor for obtaining oxygen saturation data corresponding to the user's oxygen saturation, an ECG sensor for obtaining ECG data corresponding to the user's ECG, an acceleration sensor for obtaining acceleration data corresponding to the user's movement intensity, and a radar sensor for obtaining eye movement data corresponding to the user's eye movement.

The user data may include pressure data corresponding to a pressure level for each body portion, displacement data corresponding to a displacement of the body that changes according to the user's breathing, oxygen saturation data corresponding to the user's oxygen saturation, ECG data corresponding to the user's ECG, acceleration data corresponding to the user's movement intensity, and/or eye movement data corresponding to the user's eye movement.

The plurality of sensors 5 is not limited to the above example. The plurality of sensors 5 may further include other various sensors such as an image sensor (e.g., a camera) and an audio sensor (e.g., a microphone). Furthermore, at least one of the illustrated sensors may be omitted.

The plurality of sensors 5 may each transmit the obtained user data to the hub device 1. The user data obtained by at least one of the plurality of sensors 5 may be sent to the hub device 1 through wired communication and/or wireless communication.

In an embodiment, the fourth sensor 54 may be included in a smart sensor device (e.g., a wearable device). The smart sensor device may include a wireless communication module and the fourth sensor 54. For example, the smart sensor device may include a smart watch that is shaped like a watch and/or a smart ring that is shaped like a ring, but the form of the smart sensor device is not limited thereto. The smart sensor device may establish wireless communication with the hub device 1, and transmit data collected from the fourth sensor 54 to the hub device 1 by wireless communication.

In an embodiment, the smart sensor device may include the fourth sensor 54 and the fifth sensor 55. The smart sensor device may establish wireless communication with the hub device 1, and transmit data collected from the fourth sensor 54 and the fifth sensor 55 to the hub device 1 by wireless communication.

The hub device 1 may include a memory 120 for storing a program for processing data collected from the plurality of sensors 5, and a processor 110 that is able to process the data collected from the plurality of sensors 5 based on the program stored in the memory 120. The processor 110 and the memory 120 may be provided in the plural.

The memory 120 may store a machine learning model for processing the data collected from the plurality of sensors 5. The machine learning model may be one for feature extraction, which extracts a feature of the data collected from the plurality of sensors 5 when the data is input thereto, and outputs processed data including the extracted feature. The feature of the data may include elements extracted from the data by the machine learning model to perform classification or prediction.

The machine learning model may use the data sent from the plurality of sensors 5 as an input to output the processed data including information about a state of the user.

A plurality of machine learning models corresponding to the plurality of sensors 5 may be stored in the memory 120 of the hub device 1. For example, the plurality of machine learning models may include a first machine learning model 11 for processing first data collected from the first sensor 51, a second machine learning model 12 for processing second data collected from the second sensor 52, a third machine learning model 13 for processing third data collected from the third sensor 53, a fourth machine learning model 14 for processing fourth data collected from the fourth sensor 54, and a fifth machine learning model 15 for processing fifth data collected from the fifth sensor 55.

The processor 110 of the hub device 1 may obtain first processed data by processing the first data sent from the first sensor 51. The first processed data may include feature data extracted from the first data and/or information about a state of the user extracted from the first data. The volume of the first processed data may be smaller than the volume of the first data.

The processor 110 of the hub device 1 may obtain second processed data by processing the second data sent from the second sensor 52. The second processed data may include feature data extracted from the second data and/or information about a state of the user extracted from the second data. The volume of the second processed data may be smaller than the volume of the second data.

The processor 110 of the hub device 1 may obtain third processed data by processing the third data sent from the third sensor 53. The third processed data may include feature data extracted from the third data and/or information about a state of the user extracted from the third data. The volume of the third processed data may be smaller than the volume of the third data.

The processor 110 of the hub device 1 may obtain fourth processed data by processing the fourth data sent from the fourth sensor 54. The fourth processed data may include feature data extracted from the fourth data and/or information about a state of the user extracted from the fourth data. The volume of the fourth processed data may be smaller than the volume of the fourth data.

The processor 110 of the hub device 1 may obtain fifth processed data by processing the fifth data sent from the fifth sensor 55. The fifth processed data may include feature data extracted from the fifth data and/or information about a state of the user extracted from the fifth data. The volume of the fifth processed data may be smaller than the volume of the fifth data.

The hub device 1 may primarily process the data obtained by the plurality of sensors 5 and transmit the processed data to the user device 2, thereby reducing data throughput to be borne by the user device 2. In other words, the hub device 1 may preprocess the data obtained by the plurality of sensors 5, thereby reducing the volume and/or size of the data.

The hub device 1 may include a communicator 130 including a wired communication circuit for performing wired communication with the plurality of sensors 5, and/or a wireless communication circuit for performing wireless communication with the user device 2, the server 3 and/or the home appliance. The hub device 1 may include a printed circuit board (PCB) including the processor 110, the memory 120 and the communicator 130. At least some of the plurality of sensors 5 may be wiredly connected to the PCB.

The hub device 1 may include a housing that covers the PCB. A user interface device (an input device and an output device) may be arranged in the hub device 1. The user interface may obtain a user input and output various information. The user interface is not, however, an essential element for attaining the function of the hub device 1.

The user may operate the user interface device arranged in the hub device 1 to connect the hub device 1 to an access point (AP). The user may operate the user interface device arranged in the hub device 1 to activate the communicator 130 of the hub device 1. The user may operate the user interface device arranged in the hub device 1 to power on the hub device 1.

The processor 110 of the hub device 1 may control the plurality of sensors 5. For example, the processor 110 may control at least one of the plurality of sensors 5 that is connected via wires. The processor 110 may wake up at least one of the plurality of sensors 5 connected via wires based on a sensor wakeup condition being satisfied. The waking up of the sensor may include activating the sensor.

The processor 110 may switch at least one of the plurality of sensors 5 connected via wires into a standby state based on a sensor standby condition being satisfied. The switching of the sensor into the standby state may include inactivating the sensor or operating the sensor in a low power mode.

The user device 2 may receive, from the hub device 1, data obtained by the hub device 1 by wireless communication. The data output from the hub device 1 may include processed data obtained by preprocessing the data collected from the plurality of sensors 5.

The user device 2 may include a memory 220 for storing a program for processing the preprocessed data sent from the hub device 1, and a processor 210 that is able to process the preprocessed data sent from the hub device 1 based on the program stored in the memory 220. The processor 210 and the memory 220 may be provided in the plural.

The memory 220 of the user device 2 may store a machine learning model for processing the preprocessed data sent from the hub device 1. The memory 220 of the user device 2 may store a sleep management application that is downloadable from an external server. The sleep management application is a downloadable app, which may be stored in a server of the manufacturer, a server of an application store and/or a transitory recording medium. The sleep management application may include a machine learning model. The machine learning model included in the sleep management application may be updated by the server 3.

The user device 2 may include a communicator 230 for communicating with the hub device 1, the server 3, the home appliance 4, the plurality of sensors 5 and/or a smart sensor device. The communicator 230 may include at least one of a wireless communication circuit and a wired communication circuit.

The user device 2 may receive the preprocessed data from the hub device 1 through the communicator 230. The processor 210 of the user device 2 may establish communication between the communicator 130 of the hub device 1 and the communicator 230 (e.g., a short-range wireless communication module) of the user device 2 in response to the communicator 230 being activated.

The processor 210 of the user device 2 may control a user interface 240 to request the user to activate the communicator 230 in response to the sleep management application being executed while the communicator 230 (e.g., the short-range wireless communication module) is not activated.

The processor 210 of the user device 2 may wirelessly receive data from the hub device 1 through the communicator 230 (e.g., the short-range wireless communication module).

The processor 210 of the user device 2 may use the machine learning model stored in the memory 220 to process the data received from the hub device 1. The data received from the hub device 1 may include the first processed data, the second processed data, the third processed data, the fourth processed data and/or the fifth processed data.

The processor 210 of the user deice 2 may obtain user state information relating to a state of the user by inputting the first processed data, the second processed data, the third processed data, the fourth processed data and/or the fifth processed data to the machine learning model stored in the memory 220.

The user state information may include at least one of information about a stability level of the user, information about the user's stress index, or information about the user's sleep disorder.

The machine learning model stored in the memory 220 may output the user state information relating to a state of the user when the first processed data, the second processed data, the third processed data, the fourth processed data and/or the fifth processed data are input thereto.

The processor 210 of the user device 2 may store the user state information obtained by inputting the first processed data, the second processed data, the third processed data, the fourth processed data and/or the fifth processed data to the machine learning model in the memory 220.

The processor 210 of the user device 2 may control the communicator 230 to transmit, to the server 3, the user state information obtained by inputting the first processed data, the second processed data, the third processed data, the fourth processed data and/or the fifth processed data to the machine learning model.

The communicator 230 may include a first communication module for establishing communication with the hub device 1 and a second communication module for establishing communication with the server 3. The communicator 230 may communicate with the hub device 1 in a first communication scheme and at the same time, communicate with the server 3 in a second communication scheme.

Furthermore, the user device 2 may include the user interface 240 for interacting with the user. The user interface 240 may obtain a user input. The user interface 240 may provide various information about operations of the user device 2. The user interface 240 may include an input interface and an output interface.

The input interface may include various input devices for obtaining user inputs. For example, the input interface may include a physical button, a touch screen and/or a microphone. The input interface may transmit an electric signal corresponding to the user input to the processor 210.

The user input may include various commands. For example, the user input may include a command to run the sleep management application. The sleep management application may be executed by a touch input or voice command input of the user.

The output interface may output information relating to an operation of the user device 2. The output interface may display information input by the user or information to be provided for the user in various screens. The output interface may display the information relating to the operation of the user device 2 in at least one of an image and text. Furthermore, the output interface may include a speaker for outputting sound.

The output interface may display a graphic user interface (GUI) that enables the user device 2 to be controlled. The output interface may output a GUI of the sleep management application. The output interface may display a user interface element (UI element) such as an icon.

The output interface may include a liquid crystal display (LCD) panel, light emitting diode (LED) panel, an organic LED (OLED) panel, or a micro LED panel. The output interface may include a touch display that serves as an input device as well. The output interface and the input interface may be configured separately or in one device (e.g., the touch display).

In the sleep management system 0 in the disclosure, user data obtained by the plurality of sensors 5 may be primarily processed by the hub device 1 and secondarily processed by the user device 2. The user device 2 may generate user state information and transmit the user state information to the server 3. As the user data obtained by the plurality of sensors 5 is associated with the user's privacy, it is desirable not to send the user data directly to the server 3.

In one or more embodiments, it is also possible that the hub device 1 generates the user state information by primarily processing the data obtained by the plurality of sensors 5 and secondarily processing the primarily processed data.

In one or more embodiments, it is also possible that the hub device 1 primarily processes the data obtained by the plurality of sensors 5 and transmits the primarily processed data to the server 3, and the server 3 secondarily processes the primarily processed data to generate the user state information.

In one or more embodiments, it is also possible that the hub device 1 transmits the data obtained by the plurality of sensors 5 to the user device 2, and the user device 2 primarily processes the data received from the hub device 1 and secondarily processes the primarily processed data to generate the user state information.

In one or more embodiments, it is also possible that the hub device 1 transmits the data obtained by the plurality of sensors 5 to the server 3 and the server 3 primarily processes the data received from the hub device 1 and secondarily processes the primarily processed data to generate the user state information.

The server 3 may obtain the user state information from the user device 2 through wireless communication.

The server 3 may include a memory 320 for storing a program for processing the user state information received from the user device 2, and a processor 310 that is able to process the user state information received from the user device 2 based on the program stored in the memory 320. The memory 320 may store the user state information received from the user device 2.

The server 3 may include a communicator 330 for establishing communication with the hub device 1, the user device 2, the home appliance 4 and/or the plurality of sensors 5. The communicator 330 may include at least one of a wired communication circuit and a wireless communication circuit.

The memory 320 of the server 3 may store a program for controlling the home appliance 4 based on the user state information received from the user device 2. The processor 310 of the server 3 may generate control information for controlling the home appliance 4 based on the user state information received from the user device 2. The server 3 may transmit the control information to the home appliance 4.

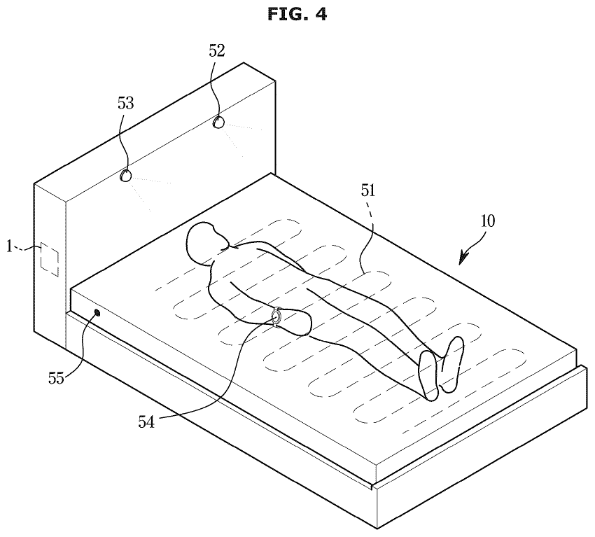

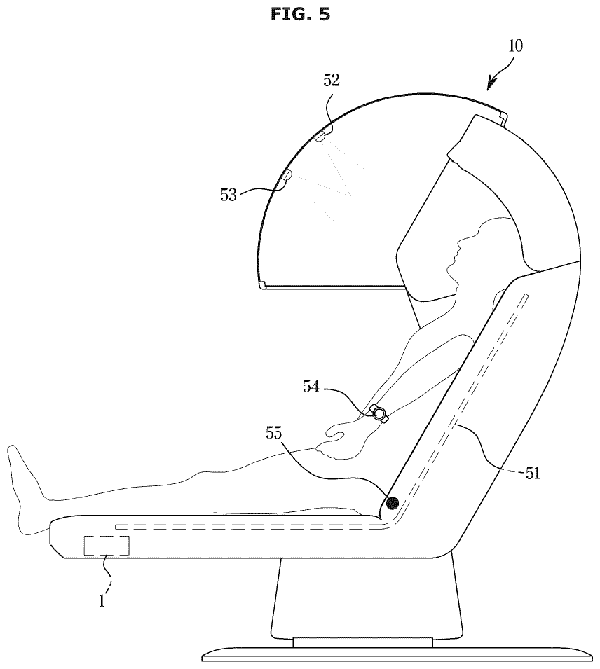

FIGS. 4 and 5 illustrate various examples of different layouts of sensors included in a sleep management system.

Referring to FIGS. 4 and 5, some of the plurality of sensors 5 (e.g., the first sensor 51, the second sensor 52 and/or the third sensor 53) may be installed on a piece of furniture 10, and the others (e.g., the fourth sensor 54 and/or the fifth sensor 55) among the plurality of sensors 5 may be arranged in a smart sensor device (e.g., a smart watch, a smart ring, etc.).

At least some of the plurality of sensors 5 may be arranged on the furniture 10 on which the user may sit or lie. The furniture 10 on which the user may sit or lie may include, for example, a bed, a chair and/or a sofa. The furniture 10 is not limited to the above example, and may have various forms that allow the user to sits or lies thereon.

The furniture 10 such as a bed, a chair or a sofa may include an actuator that is able to change the user's posture by changing its structure and/or a vibration element capable of transmitting vibration to the user.

The first sensor 51 may include a pressure sensor. The pressure sensor may include a piezoelectric element that generates an electric signal corresponding to pressure. The first sensor 51 may be installed in various locations on the furniture 10. For example, the first sensor 51 may be installed in various locations where the pressure created by the user's body portion when the user lies or sits may be measured.

When the furniture 10 corresponds to a bed, the first sensor 51 may be arranged at a mattress that comes into contact with the user's body portion (e.g., head, torso, arms, hip, or legs). The mattress may include a cover defining the exterior and having an accommodation space, and a pad arranged in the accommodation space of the cover to have the first sensor 51 placed thereon. The structure (e.g., length, width and arrangement patterns) of the first sensor 51 may vary by the size and shape of the mattress. The mattress may be placed on the floor, a chair, a sofa or a bed. The mattress may further include springs and/or sponge. The spring and/or the sponge may be arranged in the accommodation space of the cover.

When the furniture 10 corresponds to a chair, the first sensor 51 may be arranged at a seat plate, a backrest, a headrest, an arm rest and/or a leg support pressurized by the user's body portion (e.g., head, torso, arms, hip, or legs). The seat plate may contact the hip of the user. The backrest may contact the back of the user. The headrest may contact the head of the user. The armrest may contact an arm of the user. The leg support may contact the legs of the user.

The first sensor 51 may detect the pressure created by the user's body portion that comes into contact with the furniture 10. For example, the first sensor 51 may detect a distribution of the pressure created by the user who lies or sits on the furniture 10. The first sensor 51 may obtain pressure data corresponding to the pressure created by the user who lies or sits on the furniture 10.

The second sensor 52 may include a UWB sensor. The UWB sensor may include a UWB signal irradiator for transmitting a UWB signal and a UWB signal receiver for receiving a UWB signal reflecting from an object. The second sensor 52 may transmit a UWB signal to the body of the user and receive a UWB signal reflecting from the body of the user.

The second sensor 52 may have a detection region facing a body portion (e.g., torso) of the user who lies or sits on the furniture 10. The second sensor 52 may have a detection region that may detect displacement of the body caused by the user's breathing. The second sensor 52 may be arranged on the frame of the furniture 10 to have the detection region facing the body (e.g., torso) of the user, but the location of the second sensor 52 is not limited thereto.

For example, when the furniture 10 corresponds to a bed, the second sensor 52 may have a detection region facing a center portion of the bed. When the furniture 10 corresponds to a chair, the second sensor 52 may have a detection region facing a backrest of the chair.

The second sensor 52 may measure displacement of the user's body based on the UWB signal reflecting from the body of the user. For example, the second sensor 52 may measure displacement of the user's body based on a time of flight (ToF) of the UWB signal. The second sensor 52 may use the Doppler effect to measure the displacement of the user's body according to a change in wavelength (and frequency) of the UWB signal. The second sensor 52 may obtain displacement data corresponding to the displacement of the body that changes according to the user's breathing.

The third sensor 53 may include a radar sensor. The third sensor 53 may include a radar signal irradiator for transmitting a radar signal (e.g., millimeter waves or an mmWave signal) and a radar signal receiver for receiving a radar signal (e.g., an mmWave signal) reflecting from the user's body.

The frequency (e.g., 28 GHz) of the radar signal output from the third sensor 53 may be higher than the frequency (e.g., 6.0 GHZ) of the UWB signal output from the second sensor 52. A bandwidth of the radar signal output from the third sensor 53 may be narrower than the bandwidth of the UWB signal output from the second sensor 52.

The third sensor 53 may have a detection region facing a portion (e.g., face) of the body of the user who lies or sits on the furniture 10. The third sensor 53 may have a detection region that may detect a movement of eyes of the user. The third sensor 53 may measure a movement of the eyes of the user based on the mmWave reflecting from the eyes of the user. The third sensor 53 may obtain eye movement data corresponding to the movement of the eyes of the user.

The third sensor 53 may be arranged on the frame of the furniture 10 to have the detection region facing the user's body (e.g., torso), but the position of the third sensor 53 is not limited thereto. For example, when the furniture 10 corresponds to a bed, the third sensor 53 may have a detection region facing a head area of the bed. When the furniture 10 corresponds to a chair, the third sensor 53 may have a detection region facing a headrest of the chair.

The fourth sensor 54 may include an oxygen saturation sensor and/or an ECG sensor. The oxygen saturation sensor and/or the ECG sensor may include a light source for irradiating light and a photo receiver for receiving light reflecting from the user's body. The fourth sensor 54 may be arranged in a smart sensor device (e.g., a smart watch, a smart ring, etc.) that may be worn by the user. The fourth sensor 54 may operate in a non-invasive manner, irradiating light to a portion (e.g., a wrist) of the user's body and receiving light reflecting from the user's body.

The fourth sensor 54 may measure an oxygen saturation level in the user's blood and an ECG of the user based on the intensity of the light reflecting from the user's body. A portion of the light irradiated to a portion of the body may be absorbed in a blood vessel, and the oxygen saturation level in the user's blood or the user's ECG may be measured according to the light absorption rate and patterns of the absorbed light.

The fifth sensor 55 may include an acceleration sensor. The acceleration sensor may include a microelectromechanical system (MEMS) sensor, a 3-axis acceleration sensor and/or a 6-axis acceleration sensor. The fifth sensor 55 may be arranged in a smart sensor device (e.g., a smart watch, a smart ring, etc.) that may be worn by the user. The fifth sensor 55 may be installed on the furniture 10. The fifth sensor 55 may obtain acceleration data corresponding to an intensity of a movement of the user.

Types and placement sites of the plurality of sensors 5 are not limited to the above example. Many different types of sensors may be used when required, and the sensors may be installed in various locations.

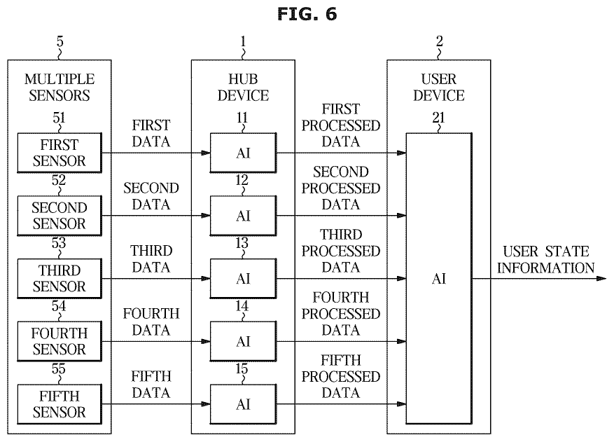

FIG. 6 illustrates a method of processing data obtained from a plurality of sensors.

Referring to FIG. 6, when the plurality of sensors 5 are activated, the hub device

1 may obtain various data from the plurality of sensors 5.

When the plurality of sensors 5 is constantly maintained in the active state, there may be a high level of power consumption by the plurality of sensors 5. The active state may refer to a state in which the plurality of sensors 5 are allowed to obtain user data by receiving the power.

The hub device 1 may activate the plurality of sensors 5 based on a preset activation condition being satisfied. At least some of the plurality of sensors 5 may be switched into the active state from an inactive state when the activation condition is satisfied. The inactive state may include a state in which power supply to the sensor is blocked or a standby state.

For example, the hub device 1 may determine whether there is a user on the furniture 10 based on the data transmitted from some of the plurality of sensors 5. The presence of a user on the furniture 10 may include the user lying or sitting on the furniture 10.

The hub device 1 may switch at least some of the plurality of sensors 5 into the standby state based on determining that there is no user on the furniture 10. For example, the hub device 1 may deactivate other sensors than the first sensor 51 and operate the first sensor 51 in a low power mode.

The operating of a sensor in the low power mode may include setting an operation period (e.g., a data collection period) of the sensor to be longer. The hub device 1 may determine whether there is a user on the furniture 10 based on data obtained by the sensor (e.g., the first sensor 51) operating in the low power mode among the plurality of sensors 5.

The hub device 1 may wake up the plurality of sensors 5 based on determining that there is a user on the furniture 10. The waking up of the plurality of sensors 5 may refer to switching the plurality of sensors 5 into the active state from the inactive state. Based on the activating of the plurality of sensors 5, the data may be transmitted to the hub device 1.

The first sensor 51 may send the first data to the hub device 1, the second sensor 52 may send the second data to the hub device 1, the third sensor 53 may send the third data to the hub device 1, the fourth sensor 54 may send the fourth data to the hub device 1, and the fifth sensor 55 may send the fifth data to the hub device 1. The plurality of sensors 5 may send the data to the hub device 1 through wired communication and/or wireless communication.

The data obtained by the plurality of sensors 5 is raw data that may have a large volume and include data related to the user's privacy. Hence, it is not desirable to directly send the raw data to the server 3. Furthermore, it may not be possible for the user device 2 to communicate with all the plurality of sensors 5, or computing capacity of the user device 2 may not be enough to process large-volume data. Such a problem may be solved by providing the hub device 1 to process the data obtained by the plurality of sensors 5.

The hub device 1 may preprocess (primary process) the raw data transmitted from the plurality of sensors 5. For this, the hub device 1 may use a machine learning model. The hub device 1 may include the first machine learning model 11 for extracting a feature from the first data transmitted from the first sensor 51, the second machine learning model 12 for extracting a feature from the second data transmitted from the second sensor 52, the third machine learning model 13 for extracting a feature from the third data transmitted from the third sensor 53, the fourth machine learning model 14 for extracting a feature from the fourth data transmitted from the fourth sensor 54, and the fifth machine learning model 15 for extracting a feature from the fifth data transmitted from the fifth sensor 55.

The first machine learning model 11 may be pretrained to extract a feature from pressure data collected by the pressure sensor. The second machine learning model 12 may be pretrained to extract a feature from displacement data collected by the UWB sensor. The third machine learning model 13 may be pretrained to extract a feature from eye-movement data collected by the radar sensor. The fourth machine learning model 14 may be pretrained to extract a feature from oxygen saturation data and/or ECG data collected by the oxygen saturation sensor and/or the ECG sensor. The fifth machine learning model 15 may be pretrained to extract a feature from acceleration data collected by the acceleration sensor.

The first machine learning model 11 may use the first data (e.g., pressure data) collected by the first sensor 51 as input data to output the first processed data. The first processed data may include, for example, information about the user's movement, posture, respiration rate and heart rate inferred by the first data. The first processed data may include information about a state of the user and information about the user's stress inferred by the first data.

The second machine learning model 12 may use the second data (e.g., displacement data) collected by the second sensor 52 as input data to output the second processed data. The second processed data may include, for example, information about a respiration rate and a heart rate inferred by the second data. The second processed data may include information about a state of the user and information about the user's stress inferred by the second data.

The third machine learning model 13 may use the third data (e.g., eye movement data) collected by the third sensor 53 as input data to output the third processed data. The third processed data may include, for example, information about an eye movement inferred by the third data. The third processed data may include information about a state of the user and information about the user's stress inferred by the third data.

The fourth machine learning model 14 may use the fourth data (e.g., oxygen saturation level data and/or ECG data) collected by the fourth sensor 54 as input data to output the fourth processed data. The fourth processed data may include, for example, information about an oxygen saturation level and/or an ECG inferred by the fourth data. The fourth processed data may include information about a state of the user and information about the user's stress inferred by the fourth data.

The fifth machine learning model 15 may use the fifth data (e.g., acceleration data) collected by the fifth sensor 55 as input data to output the fifth processed data. The fifth processed data may include, for example, information about a movement inferred by the fifth data. The fifth processed data may include information about a state of the user and information about the user's stress inferred by the fifth data.

The hub device 1 may transmit the preprocessed data to the user device 2. The data preprocessed by the hub device 1 may have a relatively small volume. Hence, data throughput to be borne by the user device 2 may be reduced.

The hub device 1 may transmit the processed data to the user device 2 by wireless communication. The communicator 130 of the hub device 1 may include a first communication circuit for receiving data collected from some (e.g., the fourth sensor 54) of the plurality of sensors 5 in a first wireless communication scheme, and a second communication circuit for transmitting the processed data to the user device 2 in a second wireless communication scheme. The first wireless communication scheme and the second wireless communication scheme may be the same or different from each other.

The user device 2 may process the processed data transmitted from the hub device 1. For this, the user device 2 may use a machine learning model. The machine learning model 21 installed in the user device 2 may include an artificial neural network (deep neural network) model with several layers (e.g., an input layer, a hidden layer, and an output layer). The machine learning model 21 installed in the user device 2 may be configured in a perceptron structure that receives multiple signals as inputs and outputs one signal. The machine learning model 21 installed in the user device 2 may be trained for estimating a state of the user based on the processed data processed by the hub device 1.

The machine learning model 21 installed in the user device 2 may use the processed data output by the machine learning model of the hub device 1 as input data to output information relating to a stability level of the user. Furthermore, the machine learning model 21 installed in the user device 2 may use the first processed data, the second processed data, the third processed data, the fourth processed data and/or the fifth processed data as input data to output user state information.

The first processed data, the second processed data, the third processed data, the fourth processed data and/or the fifth processed data may include probability information of the stability level of the user.

The first processed data may include a probability value p1 of whether a pressure level for each body portion of the user corresponds to a certain level of a plurality of levels about stability of the user.

The second processed data may include a probability value p2 of whether a respiration rate of the user corresponding to a displacement of the body corresponds to a certain level of a plurality of levels about stability of the user.

The third processed data may include a probability value p3 of whether an eye movement of the user corresponds to a certain level of a plurality of levels about stability of the user.

The fourth processed data may include a probability value p4 of whether an oxygen saturation level in the blood of the user and/or an ECG of the user corresponds to a certain level of a plurality of levels about stability of the user.

The fifth processed data may include a probability value p5 of whether a movement intensity of the user corresponds to the certain level of a plurality of levels about stability of the user.

The machine learning model 21 installed in the user device 2 may give different weights to the probability values included in the respective first to fifth processed data. The user device 2 may determine a stability level of the user by combining the probability values given the weights.

For example, factors that relatively significantly affect the determination of a stability level of the user may be the posture and movement of the user. The user device 2 may give relatively high weights to the probability value p1 included in the first processed data for pressure data and the probability value p5 included in the fifth processed data for acceleration data.

The user state information generated by the user device 2 may include the first processed data, the second processed data, the third processed data, the fourth processed data and the fifth processed data transmitted from the hub device 1. The user state information may also include information about a stability level of the user.

As such, the raw data obtained by the plurality of sensors 5 may be primarily processed by the machine learning models 11, 12, 13, 14 and 15 installed in the hub device 1 and secondarily processed by the machine learning model 21 installed in the user device 2, and thus the stability level of the user may be accurately determined.

The user device 2 may transmit the user state information to the server 3. The user device 2 may generate control information to control the home appliance 4 based on the stability level of the user, and transmit the control information to control the home appliance 4 to the server 3. The server 3 may send the control information received from the user device 2 to the home appliance 4.

The primary processing of the hub device 1 may include protection processing for privacy information of the user included in the raw data. As the user's privacy information is not transmitted directly to the server 3, the consent of the user to the collection of the user data may be obtained more easily.

The control information for controlling the home appliance 4 may be generated by the server 3. The server 3 may determine a stability level of the user based on the user state information transmitted from the user device 2, and generate control information for controlling the home appliance 4 based on the stability level of the user. The home appliance 4 may be one registered with the user account by being connected to the user device 2.

A sleep management method to help the user initiate sleep will now be described in detail.

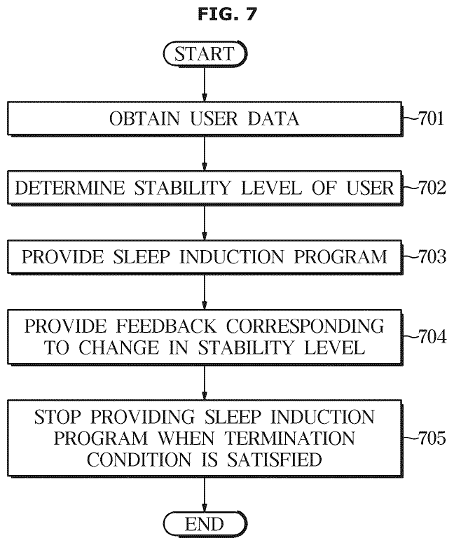

FIG. 7 is a flowchart for schematically describing an example of a sleep management method performed by a user device.

As described above, the sleep management system 0 in the disclosure may include the plurality of sensors 5, the hub device 1, and the user device 2. The plurality of sensors 5 may obtain user data. The hub device 1 may preprocess the user data obtained by the plurality of sensors 5.

Referring to FIG. 7, the user device 2 may obtain user data from the hub device 1, in 701. The user data may be preprocessed and transmitted by the hub device 1 to the user device 2.

The user device 2 may determine a stability level of the user based on the preprocessed user data, in 702. For example, the user device 2 may determine a stability level of the user based on at least one of a movement intensity of the user, a pressure level for each body portion of the user, a respiration rate of the user and a heart rate of the user included in the preprocessed user data.

The user device 2 may obtain at least one reference range regarding at least one of the movement intensity of the user, the pressure level for each body portion of the user, the respiration rate of the user and the heart rate of the user from the memory 220. In the memory 220, a first reference range for the movement intensity of the user, a second reference range for the pressure level for each body portion of the user, a third reference range for the respiration rate of the user and a fourth reference rang for the heart rate of the user may be stored in advance.

The user device 2 may determine a stability level of the user by comparing at least one of the movement intensity of the user, the pressure level for each body portion of the user, the respiration rate of the user and the heart rate of the user with at least one reference range. The stability level of the user may be classified into multiple levels. A higher level of stability may indicate that the user is in a stable state.

When the movement intensity of the user is higher than an upper limit of the first reference range, the stability of the user may be determined to have a relatively low level. On the other hand, when the movement intensity of the user is in the first reference range or lower than a lower limit of the first reference range, the stability of the user may be determined to have a relatively high level. When the user does not remain still but moves intensely, the user may be estimated to be in an unstable state.

When the pressure level for each body portion of the user is higher than an upper limit of the second reference range, the stability of the user may be determined to have a relatively low level. On the other hand, when the pressure level for each body portion of the user is in the second reference range or lower than a lower limit of the second reference range, the stability of the user may be determined to have a relatively high level.

When the user lies on the mattress of the bed, the pressure of each body portion of the user that comes into contact with the mattress may vary depending on the posture of the user. As described above, a pressure sensor may be arranged at the mattress. When the user is in an uncomfortable position (e.g., lies on a side), relatively high pressure may be applied to a certain portion (e.g., an arm and a side of the torso). When a relatively high pressure is detected from a certain body portion, the user may be estimated to be in an unstable state.

When the respiration rate of the user is higher than an upper limit of the third reference range, the stability of the user may be determined to have a relatively low level. On the other hand, when the respiration rate of the user is in the third reference range or lower than a lower limit of the third reference range, the stability of the user may be determined to have a relatively high level. In other words, when the user has a higher respiration rate, the user may be estimated to be in an unstable state.

When the heart rate of the user is higher than an upper limit of the fourth reference range, the stability of the user may be determined to have a relatively low level. On the other hand, when the heart rate of the user is in the fourth reference range or lower than a lower limit of the fourth reference range, the stability of the user may be determined to have a relatively high level. In other words, when the user has a higher heart rate, the user may be estimated to be in an unstable state.

Factors that determine a stability level of the user are not limited to the movement intensity of the user, the pressure level for each body portion of the user, the respiration rate of the user and the heart rate of the user. Apart from the aforementioned ones, other various factors may be used in determining a stability level of the user.

The user device 2 may provide a sleep induction program corresponding to the stability level of the user through the user interface 240, in 703. For example, based on the stability level of the user, the user device 2 may provide a first sleep induction program configured (i.e., designed) to guide the user to physically relax or a second sleep induction program configured (i.e., designed) to guide the user to mentally relax.

The sleep induction program may include at least one of visual information and audio information. The visual information included in the sleep induction program may be provided for the user through the display of the user interface 240. The audio information included in the sleep induction program may be provided for the user through a speaker.

The user device 2 may monitor a change in the stability level of the user according to the providing of the sleep induction program. For example, the user device 2 may monitor a change in the stability level of the user by monitoring at least one of a change in movement intensity of the user, a change in pressure level for each body portion of the user, a change in respiration rate of the user and a change in heart rate of the user while providing the seep induction program.

The user device 2 may provide feedback information corresponding to the change in the stability level of the user through the user interface 240, in 704. The feedback information may include information for guiding actions and ideas of the user. The user device 2 may monitor a change in the stability level according to the providing of the feedback information.

The user device 2 may stop providing the sleep induction program when a preset termination condition is satisfied, in 705. For example, the user device 2 may stop providing the sleep induction program based on the stability level of the user maintained to be equal to or higher than the reference level for a preset period of time.

When the stability level higher than the reference level is maintained for a certain period of time, it may be determined that the sleep of the user has begun, so the user device 2 may stop providing the sleep induction program. The condition for terminating the sleep induction program may also include an occasion when the stability level is higher than the reference level and a change in the stability level is smaller than a threshold value for a preset period of time.

The user device 2 may automatically terminate the sleep induction program even without a termination command input from the user, so the user convenience may increase.

In the meantime, the user device 2 may generate control information to control the home appliance 4 based on the stability level of the user, and transmit the control information to control the home appliance 4 to the server 3. The home appliance 4 may include at least one of a display device and an audio output device. The server 3 may send the control information received from the user device 2 to the home appliance 4. The home appliance 4 may provide the sleep induction program and feedback information in response to receiving the control information.

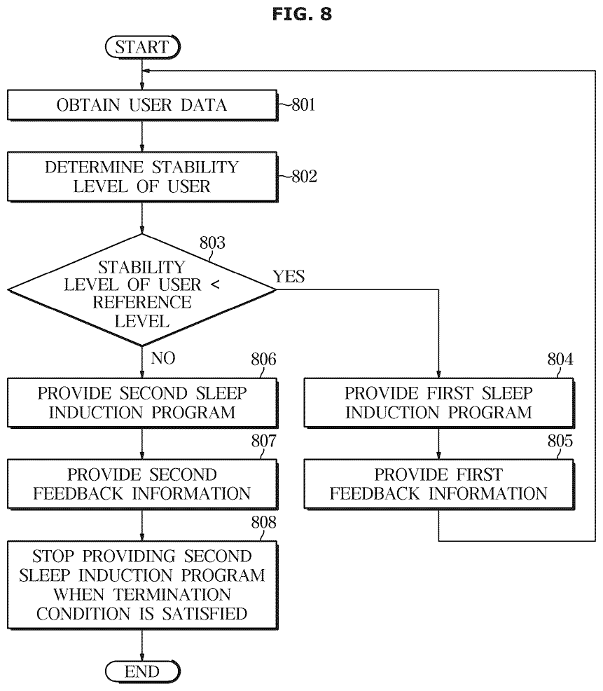

FIG. 8 is a flowchart describing the sleep management method as described in FIG. 7 in more detail.

Referring to FIG. 8, the user device 2 may obtain user data from the hub device 1, in 801. The user device 2 may determine a stability level of the user based on the obtained user data, in 802. The operations 801 and 802 may correspond to the operations 701 and 702 as described in FIG. 7.

Based on the stability level of the user, the user device 2 may provide a first sleep induction program configured to guide the user to physically relax or a second sleep induction program configured to guide the user to mentally relax. To determine whether to provide the first sleep induction program or the second sleep induction program, the user device 2 may compare the determined stability level of the user with the reference level, in 803.

The user device 2 may provide the first sleep induction program based on the stability level of the user being lower than the reference level, in 804. The reference level may be variously set for each user. That the stability level of the user is lower than the reference level may mean that the user is physically and mentally unstable or uncomfortable.

When the movement intensity of the user is higher than the upper limit of the first reference range, when the pressure level for each body portion of the user is higher than the upper limit of the second reference range, when the respiration rate of the user is higher than the upper limit of the third reference range and/or when the heart rate of the user is higher than the upper limit of the fourth reference range, the stability level may be determined to be lower than the reference level. When the stability level is lower than the reference level, the first sleep induction program may be provided first to induce the physical stability of the user.

The first sleep induction program may include at least one of visual information and audio information configured to guide the user to physically relax. For example, the first sleep induction program may correspond to a yoga program, a stretching program or a body scan program. The body scan program refers to a program that helps the user focus on his/her certain body portion.

The user device 2 may monitor a change in the stability level of the user according to the providing of the first sleep induction program, and provide first feedback information to increase the change in stability level of the user when the first sleep induction program is provided, in 805. The user is unable to sleep at a stability level of the user lower than the reference level, so the stability level needs to be quickly increased.

The first feedback information may include various information to increase the stability level by reducing movements of the user, pressure level applied to a body portion of the user, respiration rate of the user and heart rate of the user. The first feedback information may be changed in real time in response to the change in the stability level of the user.

For example, the first feedback information may include at least one of visual information and audio information for guiding movement of a certain body portion (e.g., neck, shoulder or back) of the user. The first feedback information may include at least one of image information, text information and sound information for guiding the tension of a body part to be lowered.

According to the providing of the first sleep induction program, the stability level of the user may increase. The user device 2 may provide the second sleep induction program based on the stability level of the user being equal to or higher than the reference level, in 806. That the stability level of the user is equal to or higher than the reference level may mean that the user feels stable or comfortable physically and mentally.

When the movement intensity of the user is in the first reference range or is lower than the lower limit of the first reference range, when the pressure level for each body portion of the user is in the second reference range or is lower than the lower limit of the second reference range, when the respiration rate of the user is in the third reference range or is lower than the lower limit of the third reference range and when the heart rate of the user is in the fourth reference range or is lower than the lower limit of the fourth reference range, the stability level may be determined to be higher than the reference level. When the stability level is higher than the reference level, the second sleep induction program may be provided to maintain the stability level of the user.

The second sleep induction program may include at least one of visual information and audio information configured to guide the user to mentally relax. For example, the second sleep induction program may correspond to a meditation program.

The user device 2 may monitor a change in the stability level of the user according to the providing of the second sleep induction program, and provide second feedback information to reduce the change in the stability level of the user when the second sleep induction program is provided, in 807. When the stability level of the user is equal to or higher than the reference level, maintaining the stability level constantly may help initiate falling asleep.

The second feedback information may include various information to maintain the stability at a high level by maintaining the movement of the user, pressure level applied to a body portion of the user, respiration rate of the user and heart rate of the user at low levels. The second feedback information may be changed in real time in response to the change in stability level of the user.

For example, the second feedback information may include at least one of visual information and audio information for guiding the user to do deep breathing and guiding the user to think of what makes him/her feel comfortable. As the user makes little movement and even pressure distributions are detected across the body of the user at a stability level higher than the reference level, the user device 2 may provide the second feedback information including different contents according to the change in respiration rate and heart rate of the user.

The user device 2 may stop providing the second sleep induction program when a preset termination condition is satisfied, in 808. For example, the user device 2 may stop providing the second sleep induction program based on the stability level of the user maintained to be equal to or higher than the reference level for a preset period of time. When the stability level higher than the reference level is maintained for a certain period of time, it may be determined that the sleep of the user has begun, so the user device 2 may stop providing the second sleep induction program.

The sleep management method as described in FIGS. 7 and 8 may be performed by the processor 210 of the user device 2.

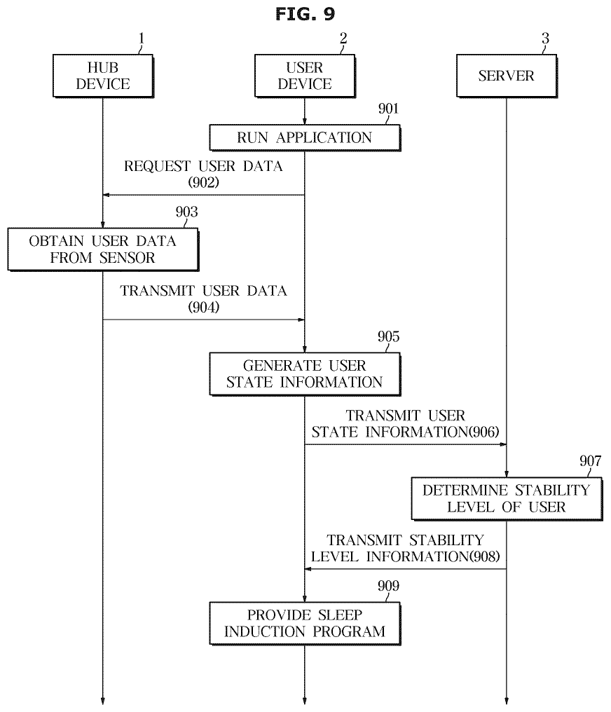

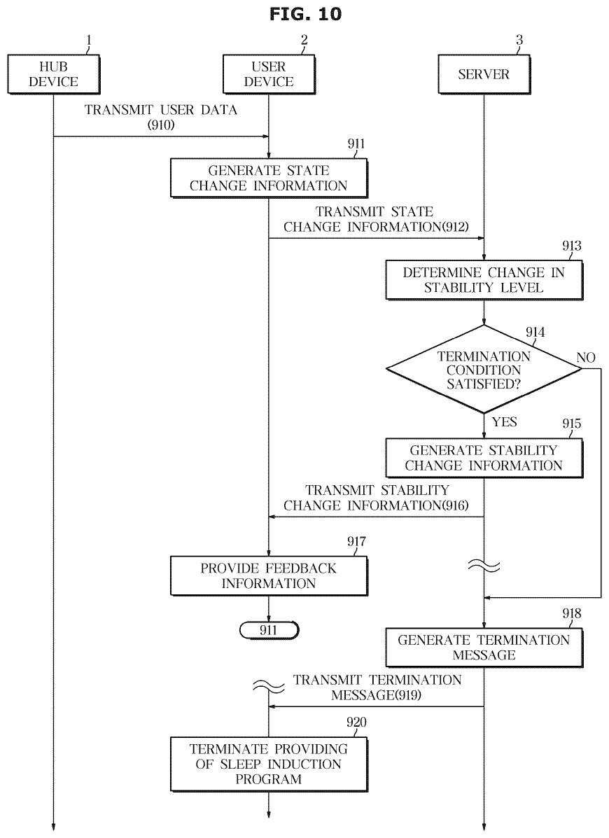

FIGS. 9 and 10 illustrate a sleep management method performed by a sleep management system.

Referring to FIG. 9, the user device 2 may run the sleep management application in response to a user input, in 901. In response to the execution of the sleep management application, the device 2 may request user data from the hub device 1, in 902. The hub device 1 may wake up the plurality of sensors 5 on receiving the request for user data from the user device 2. The hub device 1 may obtain user data from the plurality of sensors 5, in 903.

For example, the user data may include at least one of the aforementioned first data, second data, third data, fourth data and fifth data. The first data may correspond to pressure data obtained by the pressure sensor. The second data may correspond to body displacement data obtained by the UWB sensor. The third data may correspond to eye movement data obtained by the radar sensor. The fourth data may correspond to oxygen saturation data and/or ECG data obtained by the oxygen saturation sensor and/or the ECG sensor. The fifth data may correspond to acceleration data obtained by the acceleration sensor.

The hub device 1 may preprocess the user data obtained from the plurality of sensors 5 to output at least one of the first processed data, the second processed data, the third processed data, the fourth processed data and the fifth processed data. The preprocessed user data may include at least one of the first processed data, the second processed data, the third processed data, the fourth processed data and the fifth processed data.

The hub device 1 may transmit the preprocessed user data to the user device 2, in 904. The user device 2 may generate user state information based on the received user data, in 905. The user device 2 may generate the user state information by secondarily processing at least one of the first processed data, the second processed data, the third processed data, the fourth processed data and the fifth processed data generated by the hub device 1.

The user state information may include information about at least one of the movement intensity of the user, the pressure level for each body portion of the user, the respiration rate of the user and the heart rate of the user. The user state information may also include probability information for the stability level of the user.

The user device 2 may transmit the user state information to the server 3, in 906. The server 3 may determine a stability level of the user based on the user state information transmitted from the user device 2, in 907. The server 3 may generate information about the determined stability level of the user, and transmit the information about the stability level of the user to the user device 2, in 908. The information about the stability level of the user transmitted to the user device 2 from the server 3 may include information about a sleep induction program corresponding to the stability level of the user.

Although the stability level of the user is shown in FIG. 7 as being determined by the user device 2, the stability level of the user may be determined by the server 3 as shown in FIG. 8.

The user device 2 may provide a sleep induction program based on the information about the stability level of the user received from the server 3, in 909. For example, based on the stability level of the user, the user device 2 may provide a first sleep induction program configured to guide the user to physically relax or a second sleep induction program configured to guide the user to mentally relax.

FIG. 10 is continued from FIG. 9. Referring to FIG. 10, even while the user device 2 is providing the sleep induction program, the hub device 1 may transmit user data to the user device 2, in 910. In other words, while the sleep induction program is being provided, the user device 2 may receive the user data from the hub device 1 at preset intervals.

According to the providing of the sleep induction program, the user data may be changed. Specifically, while the sleep induction program is being provided, at least one of the aforementioned pressure data, body displacement data, eye movement data, oxygen saturation data, ECG data and acceleration data may be changed.

The user device 2 may generate state change information based on the user data transmitted from the hub device 1 being changed, in 911. The stage change information may include information about a change in state of the user. For example, the state change information may include information about at least one of a change in movement intensity of the user, a change in pressure level for each body portion of the user, a change in respiration rate of the user and a change in heart rate of the user.

The user device 2 may transmit the state change information to the server 3, in 912. The server 3 may determine a change in the stability level of the user based on the state change information transmitted from the user device 2, in 913.

The server 3 may determine whether the termination condition for terminating the providing of the sleep induction program is satisfied, in 914. For example, the server 3 may determine whether the stability level of the user is maintained to be equal to or higher than the reference level for a preset period of time.

When the stability level higher than the reference level is maintained for a certain period of time, it may be determined that the sleep of the user has begun, so the server 3 may determine to stop providing the sleep induction program. The condition for terminating the sleep induction program may also include an occasion when the stability level is higher than the reference level and a change in the stability level is smaller than a threshold value for a preset period of time.

When the termination condition for terminating the providing of the sleep induction program is not satisfied, the server 3 may generate stability change information in 915 and transmit the stability change information to the user device 2 in 916.

The user device 2 may provide feedback information corresponding to the stability change information, in 917. For example, the user device 2 may provide first feedback information to increase a change in the stability level of the user when the first sleep induction program is provided. The user device 2 may provide second feedback information to reduce a change in the stability level of the user when the second sleep induction program is provided.

The user device 2 may monitor a change in the state of the user corresponding to the providing of the feedback information based on the user data transmitted from the hub device 1. After the feedback information is provided, obtaining of user data, generating of state change information and determining of a change in the stability level may be performed again.

When the termination condition for terminating the providing of the sleep induction program is satisfied, the server 3 may generate a termination message for terminating the providing of the sleep induction program, in 918. The server 3 may transmit the termination message to the user device 2, in 919. The user device 2 may stop providing the sleep induction program based on the termination message being received from the server 3, in 920.

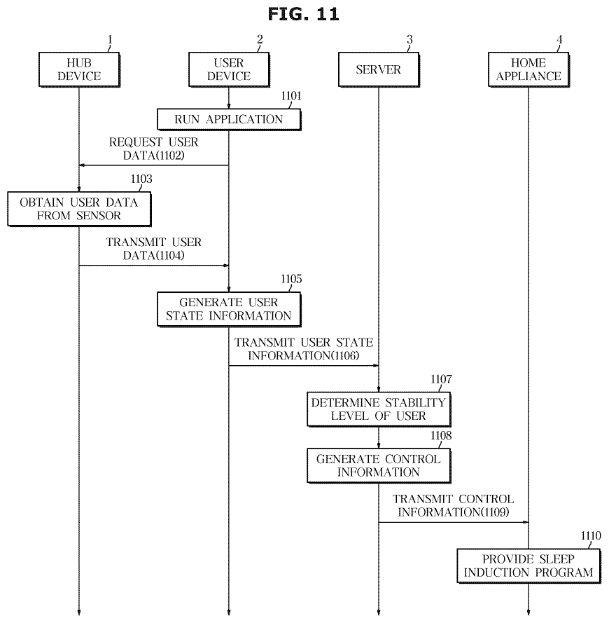

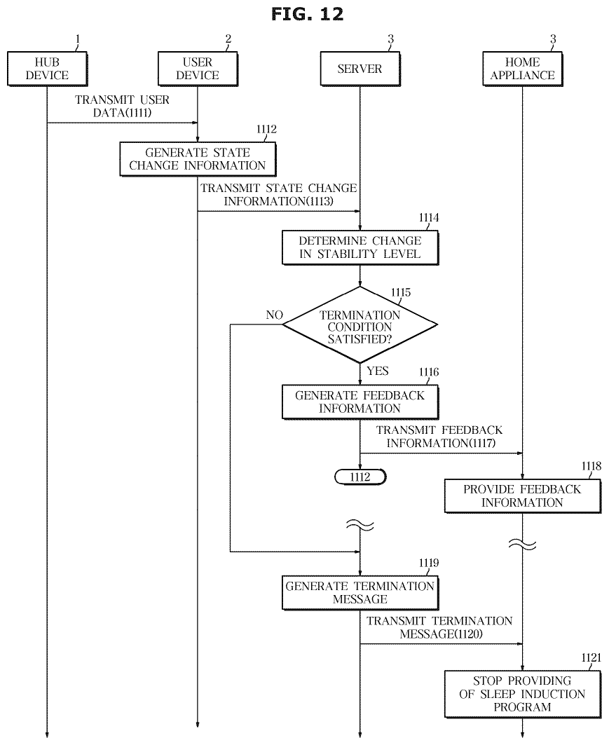

FIGS. 11 and 12 illustrate a sleep management method performed by a sleep management system, according to another embodiment.

Referring to FIG. 11, operations 1101, 1102, 1103, 1104, 1105, 1106 and 1107 may correspond to operations 901 to 907 as described in FIG. 9.

The server 3 may generate control information for controlling the home appliance 4, in 1108. The control information for controlling the home appliance 4 may be generated based on the user state information and stability level of the user received from the user device 2. The server 3 may transmit the generated control information to the home appliance 4, in 1109.

The home appliance 4 may provide a sleep induction program based on the control information being received from the server 3, in 1110. For example, the home appliance 4 may include at least one of a display device and an audio output device. The display device may output at least one of visual information and audio information provided by the sleep induction program. The audio output device may output audio contents provided by the sleep induction program.

FIG. 12 is continued from FIG. 11. Referring to FIG. 12, even while the home appliance 4 is providing the sleep induction program, the hub device 1 may transmit user data to the user device 2, in 1111. The user device 2 may generate state change information based on the user data transmitted from the hub device 1 being changed, in 1112. The user device 2 may transmit the state change information to the server 3, in 1113.

The server 3 may determine a change in stability level of the user based on the state change information transmitted from the user device 2, in 1114. The server 3 may determine whether the termination condition for terminating the providing of the sleep induction program is satisfied, in 1115.

When the termination condition for terminating the providing of the sleep induction program is not satisfied, the server 3 may generate feedback information corresponding to the change in the stability level of the user, in 1116. The server 3 may transmit the generated feedback information to the home appliance 4, in 1117. The home appliance 4 may provide the feedback information received from the server 3 to the user, in 1118.

For example, the server 3 may generate first feedback information to increase a change in the stability level of the user when the first sleep induction program is being provided through the home appliance 4. The server 3 may generate second feedback information to reduce a change in the stability level of the user when the second sleep induction program is being provided through the home appliance 4. The home appliance 4 may output the first feedback information or the second feedback information.

The user device 2 may monitor a change in state of the user corresponding to the providing of the feedback information based on the user data transmitted from the hub device 1. After the feedback information is provided, obtaining of user data, generating of state change information and determining of a change in stability level may be performed again.

When the termination condition for terminating the providing of the sleep induction program is satisfied, the server 3 may generate a termination message for terminating the providing of the sleep induction program, in 1119. The server 3 may transmit the termination message to the home appliance 4, in 1120. The home appliance 4 may stop providing the sleep induction program based on the termination message being received from the server 3, in 1121.

As described above in connection with FIGS. 7 to 12, the sleep management system and sleep management method in the disclosure may automatically provide a sleep induction program suitable for the user. The sleep management system and sleep management method in the disclosure may provide proper feedback by monitoring the stability level of the user and automatically provide and terminate the sleep induction program. Accordingly, they may provide effective help for sleep initiation of the user.

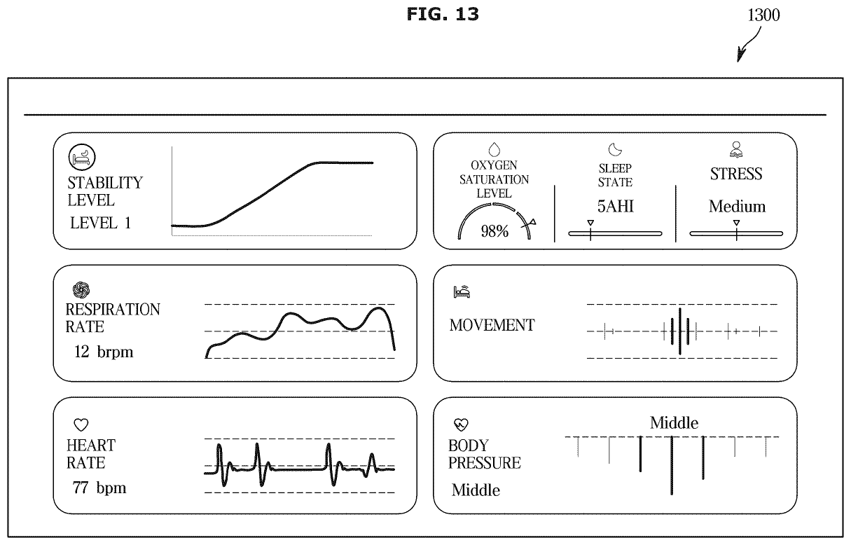

FIG. 13 illustrates an example of a screen 1300 for user state information provided through at least one of a user device and a home appliance.

The user state information may be generated by the sleep management system in real time. In other words, the user state information may be generated at preset time intervals.

Referring to FIG. 13, the user state information may include information about a stability level, information about oxygen saturation, information about sleep disorder, information about a stress index, information about a respiration rate, information about a movement, information about a heart rate and information about body pressure. The user state information may be provided through the user device 2 or the home appliance 4.

The information about the stability level may include information about a current stability level of the user and a change in stability level. The information about the stability level may be obtained by the user with the user device 2 at a preset point in time, a point in time at which the user lies or sits on the furniture 10 for sleep, or a point in time when the user runs the sleep management application. The information about the stability level may be represented in the form of a graph, the x-axis of which may represent time and the y-axis of which may represent the stability level of the user.

The information about oxygen saturation may include information about current oxygen saturation of the user and information about the user's oxygen saturation over time. When the information about oxygen saturation is output through the user device 2 or the home appliance 4, numerical values of the oxygen saturation may be output in percentage. Furthermore, when the information about oxygen saturation is output by the user device 2 or the home appliance 4, whether the oxygen saturation of the user is normal according to medical standards may be displayed.

The information about a sleep state may include information whether the user is in an awake state, a rapid eye movement (REM) sleep state or a non REM (NREM) state.

The information about a stress index may include information about a stress level of the user. When the information about a stress index is output through the user device 2 or the home appliance 4, the user's stress index may be output in a numerical value or in the form of a comparative word (e.g., high, medium, and low) with an indication whether the user's stress index is normal according to the medical standard.

The information about a respiration rate may include information about a current respiration rate of the user and information about the user's respiration rates over time. When the information about the current respiration rate is output through the user device 2 or the home appliance 4, the user's respiration rate may be output in a numerical value with an indication whether the user's respiration rate is normal according to the medical standard.

The information about a movement may include information about an intensity of the user's movement and information about the user's movement intensities over time. When the information about the current movement degree is output through the user device 2 or the home appliance 4, the user's movement degree may be output in a numerical value or in the form of a comparative word (e.g., high, medium, and low).

The information about a heart rate may include information about a current heart rate of the user and information about the user's heart rates over time. When the information about the current heart rate is output through the user device 2 or the home appliance 4, the user's heart rate may be output in a numerical value with an indication whether the user's heart rate is normal according to the medical standard.

The information about body pressure may include information about a pressure level for each body portion of the user. Furthermore, the information about the body pressure may include information about a posture of the user. For example, the information about the body pressure may include information regarding whether the user's posture corresponds to lying on one's back, on the left side, on the right side, or on one's face, sitting up, etc.

When the information about the body pressure is output through the user device 2 or the home appliance 4, a term that indicates the posture of the user may be used, and pressure distribution information across the body of the user may be provided.

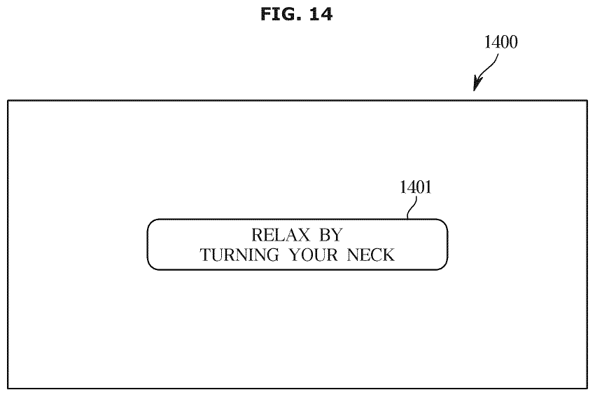

FIG. 14 illustrates an example of feedback information provided based on a stability level of a user when a sleep induction program is provided. Referring to FIG. 14, an example of a screen 1400 displayed through the user device 2 or the home appliance 4 is illustrated.

When the stability level of the user is lower than the reference level, the first sleep induction program may be provided to guide the user to physically relax. For example, the first sleep induction program may correspond to a yoga program, a stretching program or a body scan program.

While the first sleep induction program is being provided, the first feedback information according to a change in the stability level of the user may be displayed. The user is unable to sleep at a stability level of the user lower than the reference level, so the stability level needs to be quickly increased.

The first feedback information may include various information to increase the stability level by reducing movements of the user, pressure level applied to a body portion of the user, respiration rate of the user and heart rate of the user. The first feedback information may include visual information for increasing a change in the stability level of the user.

For example, the first feedback information may include at least one of image information and text information for guiding movement of a certain body portion (e.g., neck, shoulder or back) of the user. As shown in FIG. 14, a text message 1401 such as “relax by turning your neck” may be displayed through the user device 2 or the home appliance 4. The first feedback information may be changed in real time in response to the change in the stability level of the user. Furthermore, as described above, the first feedback information may be provided as audio information such as voice instructions.

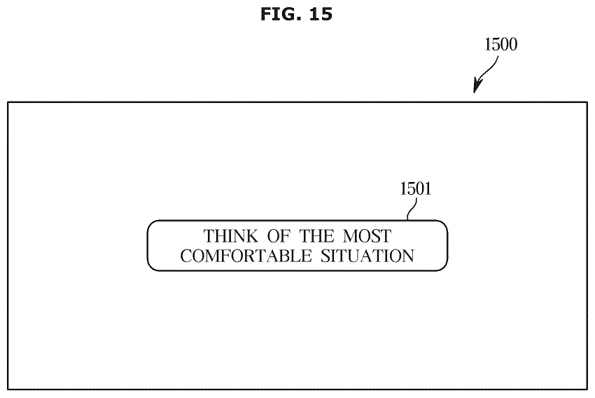

FIG. 15 illustrates another example of feedback information provided based on a stability level of a user when a sleep induction program is provided. Referring to FIG. 15, another example of a screen 1500 displayed through the user device 2 or the home appliance 4 is illustrated.

When the stability level of the user is equal to or higher than the reference level, the second sleep induction program may be provided to guide the user to mentally relax. For example, the second sleep induction program may correspond to a meditation program.

The second feedback information may include various information to maintain the stability at a high level by maintaining the movement of the user, pressure level applied to a body portion of the user, respiration rate of the user and heart rate of the user at low levels.

For example, the second feedback information may include at least one of image information and text information for guiding the user to do deep breathing and guiding the user to think of what makes him/her feel comfortable. As shown in FIG. 15, a text message 1501 such as “think of the most comfortable situation” may be displayed through the user device 2 or the home appliance 4. The second feedback information may be changed in real time in response to the change in the stability level of the user. Furthermore, as described above, the second feedback information may be provided as audio information such as voice instructions.

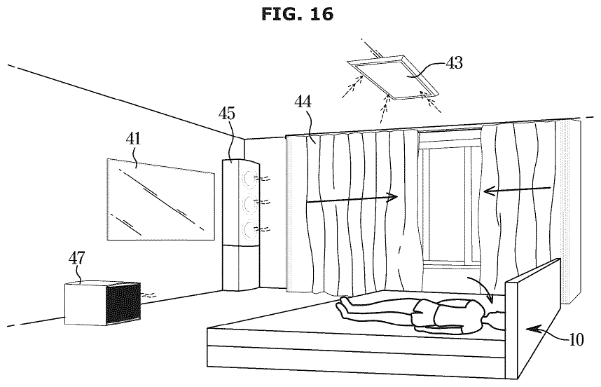

FIGS. 16, 17 and 18 illustrate various examples of controlling home appliances to provide a sleep induction program and feedback information.

Referring to FIG. 16, the server 3 may control the display device 41 to perform a certain operation to induce the user to fall sleep. For example, the server 3 may control the display device 41 so that the brightness of an image output from the display device 41 gradually becomes darker. The server 3 may control the display device 41 to play preset music (e.g., music to induce sleep).

The server 3 may control the speaker 46 to play certain music (e.g., music to induce sleep).

The server 3 may control the lighting device 43 to perform a preset operation to induce sleep. For example, the server 3 may control the lighting device 43 so that the brightness of light output from the lighting device 43 gradually becomes darker. The server 3 may control the lighting device 43 so that color of the light output from the lighting device 43 is changed to a preset color (e.g., a color having a color temperature of light of 2000K or less).

The server 3 may control the automatic curtain open/close device 44 to perform a preset operation to induce sleep. For example, the server 3 may control the automatic curtain open/close device 44 to close the curtain.

The server 3 may control the air conditioner 45 and/or the air purifier 47 to perform a preset operation to induce sleep. For example, the server 3 may control the air conditioner and/or the air purifier 47 to operate in a sleep mode. While operating in the sleep mode, the air conditioner 45 and/or the air purifier 47 may minimize noise, for example, by controlling the fan speed to slow down.

The preset operation to induce sleep that may be performed by the home appliance 4 is not limited to the above example, and may be changed according to the user's setting and the type of the home appliance 4.

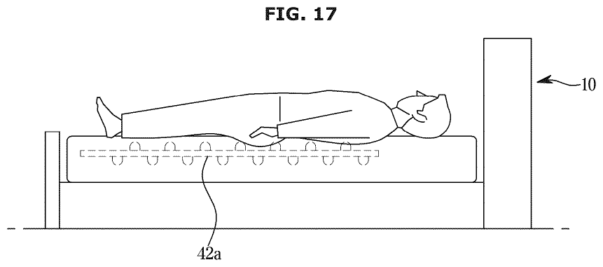

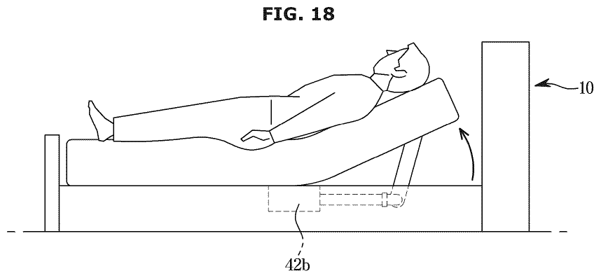

Referring to FIGS. 17 and 18, the furniture control device 42 may include the vibration element 42a that is able to transmit vibration to the user who lies or sits on the furniture 10 and/or the actuator 42b that is able to change the posture of the user by changing the structure of the furniture 10.

The server 3 may control the furniture control device 42 to perform a preset operation to induce sleep. For example, the server 3 may control the vibration element 42a to output vibration at preset intervals to ease the tension of the user. The server 3 may control the actuator 42b to change the structure of the furniture so that the user's posture is changed to a preset posture (lying on his/her back) that makes it easy to fall sleep.

The sleep management system and sleep management method in the disclosure may automatically provide a sleep induction program suitable for the user.

The sleep management system and sleep management method in the disclosure may provide proper feedback by monitoring the stability level of the user and automatically provide and terminate the sleep induction program. Accordingly, they may provide effective help for sleep initiation of the user.

The embodiments of the disclosure may be implemented in the form of a recording medium for storing instructions to be carried out by a computer. The instructions may be stored in the form of program codes, and when executed by a processor, may generate program modules to perform operations in the embodiments of the disclosure. The recording media may correspond to computer-readable recording media.

The computer-readable recording medium includes any type of recording medium having data stored thereon that may be thereafter read by a computer. For example, it may be a read only memory (ROM), a random access memory (RAM), a magnetic tape, a magnetic disk, a flash memory, an optical data storage device, etc.

The computer-readable storage medium may be provided in the form of a non-transitory storage medium. The term “non-transitory storage medium” may mean a tangible device without including a signal, e.g., electromagnetic waves, and may not distinguish between storing data in the storage medium semi-permanently and temporarily. For example, the non-transitory storage medium may include a buffer that temporarily stores data.

In an embodiment of the disclosure, the aforementioned method according to the one or more embodiments of the disclosure may be provided in a computer program product. The computer program product may be a commercial product that may be traded between a seller and a buyer. The computer program product may be distributed in the form of a recording medium (e.g., a compact disc read only memory (CD-ROM)), through an application store (e.g., Play store™), directly between two user devices (e.g., smart phones), or online (e.g., downloaded or uploaded). In the case of online distribution, at least part of the computer program product (e.g., a downloadable app) may be at least temporarily stored or arbitrarily created in a recording medium that may be readable to a device such as a server of the manufacturer, a server of the application store, or a relay server.

Several embodiments of the disclosure have been described above, but a person of ordinary skill in the art will understand and appreciate that various modifications can be made without departing from the scope of the disclosure. Thus, it will be apparent to those of ordinary skill in the art that the true scope of technical protection is only defined by the following claims.

What is claimed is:

1. A sleep management system comprising:
a sensor configured to collect data regarding a user;
a hub device configured to preprocess the data obtained by the sensor; and
a user device comprising: at least one memory storing one or more instructions; and at least one processor configured to execute the one or more instructions,
wherein the one or more instructions, when executed by the at least one processor of the user device, are configured to cause the user device to:
obtain the preprocessed data from the hub device,
identify a stability level of the user based on the preprocessed data,
provide, through a user interface, a sleep induction program corresponding to the stability level of the user,
monitor a change in the stability level of the user contemporaneously with the provision of the sleep induction program, and
provide, through the user interface, feedback information corresponding to the change in the stability level of the user.

2. The sleep management system of claim 1, wherein the one or more instructions, when executed by the at least one processor of the user device, are further configured to cause the user device to:
provide the sleep induction program by providing, based on the stability level of the user, a first sleep induction program configured to guide the user to physically relax or a second sleep induction program configured to guide the user to mentally relax.

3. The sleep management system of claim 2, wherein the one or more instructions, when executed by the at least one processor of the user device, are further configured to cause the user device to:
based on the stability level of the user being lower than a reference level, provide the first sleep induction program, and
based on the stability level of the user being equal to or higher than the reference level, provide the second sleep induction program.

4. The sleep management system of claim 3, wherein the one or more instructions, when executed by the at least one processor of the user device, are further configured to cause the user device to:
stop providing the second sleep induction program based on the stability level of the user being equal to or higher than the reference level for a preset period of time.

5. The sleep management system of claim 2, wherein on the preprocessed data comprises at least one of a movement intensity of the user, a pressure level of a body portion of the user, a respiration rate of the user, and a heart rate of the user.

6. The sleep management system of claim 5, wherein the one or more instructions, when executed by the at least one processor of the user device, are further configured to cause the user device to:
monitor the change in the stability level of the user based on the first sleep induction program or the second sleep induction program being provided by monitoring a change in at least one of the movement intensity, the pressure level, the respiration rate and the heart rate.

7. The sleep management system of claim 6, wherein the one or more instructions, when executed by the at least one processor of the user device, are further configured to cause the user device to:
based on the first sleep induction program being provided, provide first feedback information configured to increase the change in the stability level of the user, and
based on the second sleep induction program being provided, provide second feedback information configured to reduce the change in the stability level of the user.

8. The sleep management system of claim 5, wherein the one or more instructions, when executed by the at least one processor of the user device, are further configured to cause the user device to:
obtain at least one reference range regarding at least one of the movement intensity of the user, the pressure level for the body portion of the user, the respiration rate of the user and the heart rate of the user from at least one memory, and
identify the stability level of the user by comparing at least one of the movement intensity, the pressure level, the respiration rate and the heart rate with the at least one reference range.

9. The sleep management system of claim 1, wherein the one or more instructions, when executed by the at least one processor of the user device, are further configured to cause the user device to:
control the user interface to provide the sleep induction program and the feedback information as at least one of visual information and audio information.

10. The sleep management system of claim 1, wherein the one or more instructions, when executed by the at least one processor of the user device, are further configured to cause the user device to:
generate control information for providing the sleep induction program and the feedback information through at least one of a display device and an audio output device, and transmit the control information to a server.

11. A sleep management method comprising:
obtaining data regarding a user through a sensor;
preprocessing the data by a hub device;
identifying, by a user device, a stability level of the user based on the preprocessed data;
providing, by the user device, a sleep induction program corresponding to the stability level of the user;
monitoring, by the user device, a change in the stability level of the user contemporaneously with the providing the sleep induction program; and
providing, by the user device, feedback information corresponding to the change in the stability level of the user.

12. The sleep management method of claim 11, wherein the providing the sleep induction program comprises providing, based on the stability level of the user, a first sleep induction program configured to guide the user to physically relax or a second sleep induction program configured to guide the user to mentally relax.

13. The sleep management method of claim 12, wherein the providing the sleep induction program further comprises:
based on the stability level of the user being lower than a reference level, providing the first sleep induction program; and
based on the stability level of the user being equal to or higher than the reference level, providing the second sleep induction program.

14. The sleep management method of claim 13, further comprising:
based on the stability level of the user maintained being equal to or higher than the reference level for a preset period of time, terminating the providing of the second sleep induction program.

15. The sleep management method of claim 12, wherein the preprocessed data comprises at least one of a movement intensity of the user, a pressure level of a body portion of the user, a respiration rate of the user, and a heart rate of the user.

16. A non-transitory computer readable medium having instructions stored therein, which when executed by at least one processor cause the at least one processor to execute a sleep management method, the sleep management method comprising:
obtaining data regarding a user through a sensor;
preprocessing the data by a hub device;
identifying, by a user device, a stability level of the user based on the preprocessed data;
providing, by the user device, a sleep induction program corresponding to the stability level of the user;
monitoring, by the user device, a change in the stability level of the user contemporaneously with the providing the sleep induction program; and
providing, by the user device, feedback information corresponding to the change in the stability level of the user.

17. The non-transitory computer readable medium of claim 16, wherein the providing the sleep induction program comprises providing, based on the stability level of the user, a first sleep induction program configured to guide the user to physically relax or a second sleep induction program configured to guide the user to mentally relax.

18. The non-transitory computer readable medium of claim 17, wherein the providing the sleep induction program further comprises:
based on the stability level of the user being lower than a reference level, providing the first sleep induction program; and
based on the stability level of the user being equal to or higher than the reference level, providing the second sleep induction program.

19. The non-transitory computer readable medium of claim 18, where the sleep management method further comprises:
based on the stability level of the user maintained being equal to or higher than the reference level for a preset period of time, terminating the providing of the second sleep induction program.

20. The non-transitory computer readable medium of claim 17, wherein the preprocessed data comprises at least one of a movement intensity of the user, a pressure level of a body portion of the user, a respiration rate of the user, and a heart rate of the user.
